(19) United States
(12) Patent Application Publication
(10) Pub. No.: US 2011/0091422 A1
(43) Pub. Date: Apr. 21, 2011

(54) Use of Genetic Markers for Identifying the Response to Interferon Treatment in Multiple Sclerosis Patients

(75) Inventors: Emmanuel Monnet, Saint Julien en Genevois (FR); Nourdine Bouzekri, Vetraz-Monthoux (FR)

(73) Assignee: MERCK SERONO SA, COINSINS, VAUD (CH)

(21) Appl. No.: 12/994,181

(22) PCT Filed: Jul. 20, 2009
(86) PCT No.: PCT/EP2009/059283

§ 371 Date: Nov. 23, 2010

Related U.S. Application Data
(60) Provisional application No. 61/135,970, filed on Jul. 25, 2008.

(30) Foreign Application Priority Data
Jul. 24, 2008 (EP) 08104874.6

(51) Int. Cl. A61K 38/21; C12Q 1/68; A61P 29/00

(52) U.S. Cl. 424/85.6; 435/6

(57) ABSTRACT

The present invention relates to the use of genetic markers to identify the response to interferon-beta (IFN-beta) treatment in Multiple Sclerosis (MS) patients as well as a method for treating MS patients and kits for genotyping.

### FIELD OF THE INVENTION

The present invention relates to the use of genetic markers to identify response to interferon treatment, in particular interferon-beta (IFN-beta) treatment, in Multiple Sclerosis (MS) patients, a method for treating a patient group suffering from MS as well as kits for genotyping.

### BACKGROUND OF THE INVENTION

Multiple Sclerosis (MS) is one of the most common neurological diseases. It is an autoimmune condition in which the immune system attacks the central nervous system leading to demyelination. It may cause numerous physical and mental symptoms, and often progresses to physical and cognitive disability. Disease onset usually occurs in young adults and has a prevalence that ranges between 2 and 150 per 100,000 depending on the country or specific population. One can distinguish various forms of MS.

Relapsing-remitting MS (RRMS): Relapsing-remitting describes the initial course of 85% to 90% of individuals with MS. This subtype is characterized by unpredictable attacks (relapses) followed by periods of months to years of relative quiet periods with no new signs of disease activity. Deficits suffered during the attacks may either resolve or may be permanent.

Secondary progressive MS (SPMS): Secondary progressive describes around 80% of those with initial relapsing-remitting MS, who then begin to have neurologic decline between their acute attacks without any definite periods of remission. This decline may include new neurologic symptoms, worsening cognition function, or other deficits. Secondary progressive is the most common type of MS.

Primary progressive MS (PPMS): Primary progressive describes the approximately 10% of individuals who never have remission after their initial MS symptoms. Decline occurs continuously without clear attacks. The primary progressive subtype tends to affect people who are older at disease onset.

Progressive relapsing MS (PRMS): Progressive relapsing describes those individuals who, from the onset of their MS, have a steady neurologic decline but also suffer superimposed attacks. This type of MS is the least common of all subtypes.

Various disease modifying medications have been developed. One approach uses interferon-beta (IFN-beta) existing in various forms. IFN-beta can also be distinguished according to its production process in eukaryotes or prokaryotes. It is marketed inter alia as Rebif®, Avonex®, Cinnovex® or Betaseron®. MS patients show different levels of response to the treatment.

In recent years pharmacogenomics—also denoted pharmacogenetics—(PGx) has come into focus of physicians. It can be viewed as the study of clinical testing of genetic variation of individuals that gives rise to differing responses to drugs. In this context the genome of an individual may be analyzed. Biomarkers or genetic markers are of significance in this regard.

A genetic marker is a known DNA sequence. It can be described as a variation, which may arise due to mutation or alteration in the genomic loci that can be observed. A genetic marker is e.g. a DNA sequence, such as a sequence surrounding a single base-pair change, i.e. a single nucleotide polymorphism (SNP).

Pharmacogenomics and genetic markers are applied in approaches of personalized medicine. Personalized medicine is the use of information and data from e.g. a patient's genotype to stratify disease, select a medication, provide a therapy, or initiate a preventative measure that is particularly suited to that patient at the time of administration. In addition to genetic information, other factors, including imaging, laboratory, and clinical information about the disease process or the patient play an equally important role. It is believed that personalized medicine will make it possible in the future to give the most appropriate drug, at preferably an optimum dose, preferably to the most specific patient group, at the optimum time.

Cunnigham et al. (1) investigated genetic markers indicative of a response to interferon-beta treatment in Multiple Sclerosis patients. Neither IFNAR2 nor a combination of IFNAR2 with PSMB8 and MX-1, respectively, is disclosed therein. Such combinations are also not suggested in this publication.

Sugimoto et al. (2) describe LMP7-K (also called PSMB8) and the response to interferon-alpha treatment in hepatitis C patients.

Leyva et al. (3) published IFNAR1 and IFNAR2 (SNP 11876 or rs1051393) genes in Multiple Sclerosis susceptibility and in interferon-beta treatment response in patients. It was reported that variations in IFNAR1 and IFNAR2 genes were not relevant to the interferon-beta therapy responsiveness.

### SUMMARY OF THE INVENTION

According to one aspect of the invention, is provided a method of identifying response upon treatment with interferon in an individual having Multiple Sclerosis (MS) by use of genotyping. In particular the response of a MS patient upon interferon-beta treatment is an aspect of the application.

According to another aspect of the invention, is provided a method for treating MS comprising genotyping the MS patient and based on the genotyping result treating the MS patient.

According to another aspect of the invention, is provided a kit for detecting a genetic marker that is associated with response to interferon, in particular the response, in an individual having Multiple Sclerosis (MS).

### BRIEF DESCRIPTION OF THE DRAWINGS

FIG. 1 describes the marker IFNAR2-rs4986956 (also denoted rs2229207) and the response to interferon-beta 1a (Rebif®) treatment in MS patients.

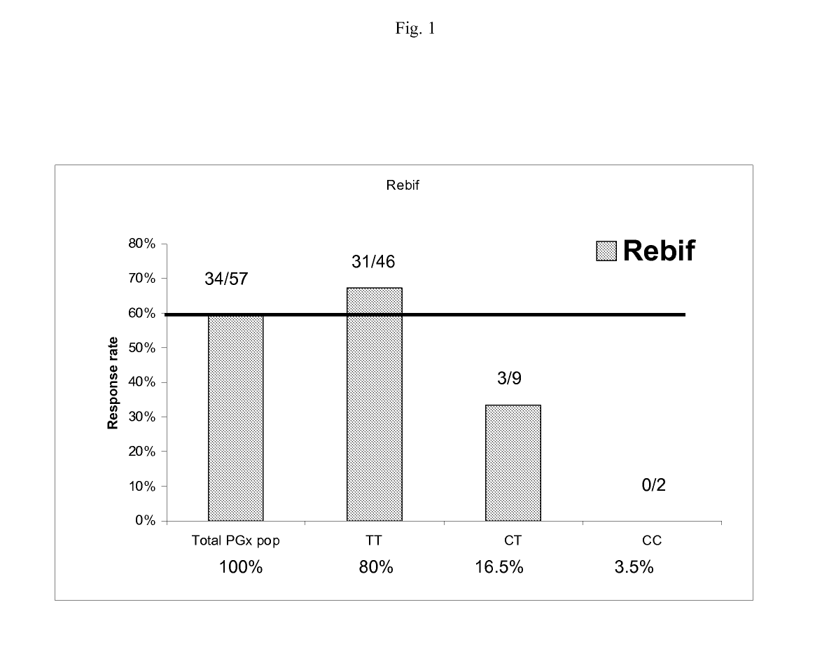

FIG. 2 describes the marker PSMB8-rs2071543 and the response to interferon-beta 1a (Rebif®) treatment in MS patients.

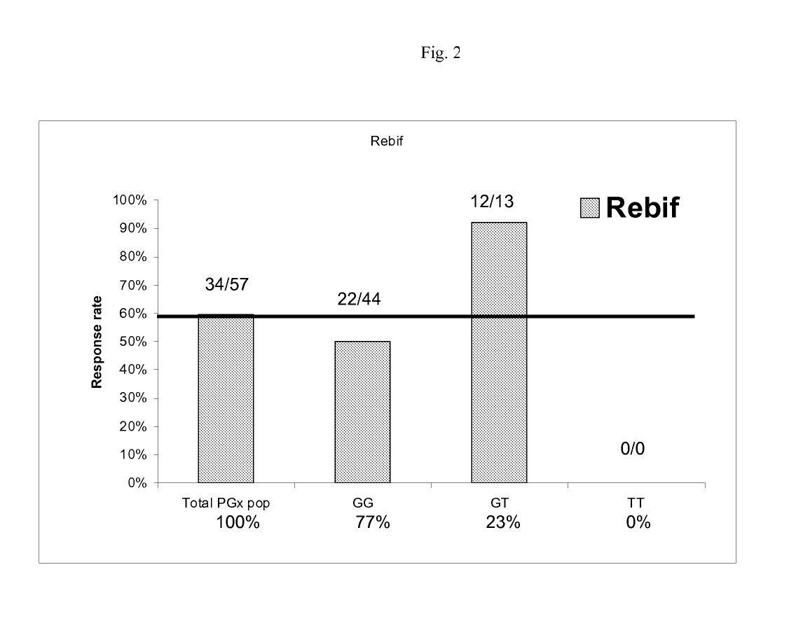

FIG. 3 describes the marker MX1-rs469304 and the response to interferon-beta 1a (Rebif®) treatment in MS patients.

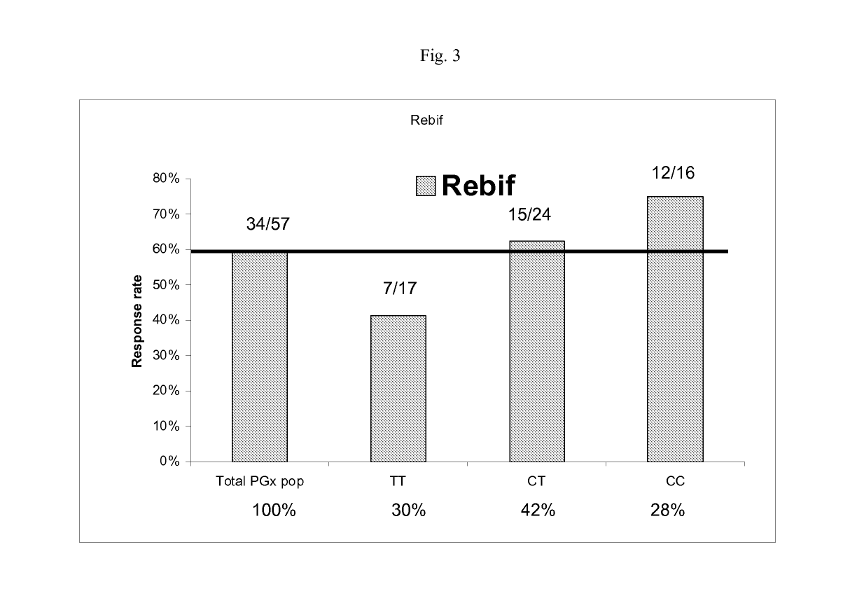

FIG. 4 describes the combination of markers IFNAR2-rs4986956 and PSMB8-rs2071543 and the response to interferon-beta 1a (Rebif®) treatment in MS patients.

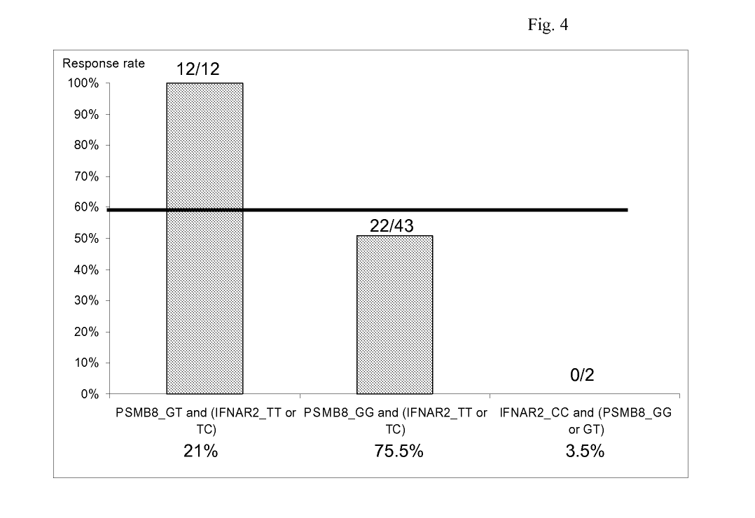

### DETAILED DESCRIPTION OF THE INVENTION

The present invention is directed to a method of identifying response, particularly the level of response, upon treatment with interferon, preferably interferon-beta, most preferably interferon-beta 1a, in an individual having Multiple Sclerosis (MS), the method comprising the steps: a. obtaining a DNA sample of said individual; b. determining whether in IFNAR2-rs4986956 a tyrosine or a cytosine is present; or in IFNAR2-rs4986956 a tyrosine or a cytosine and in PSMB8-rs2071543 a guanosine or a tyrosine is present; or in IFNAR2-rs4986956 a tyrosine or a cytosine and in MX1-rs469304 a tyrosine or a cytosine is present as biallelic markers; c. correlating the result of step b. with the level of response to the treatment with interferon-beta.

The invention is based on genetic tests measuring the presence or absence of genetic markers called single nucleotide polymorphism (SNP) and predicting the response to interferon treatment, preferably to interferon-beta treatment, in Multiple Sclerosis (MS) patients. Preferably the interferon is an interferon-beta 1a or 1b, more preferably Rebif®, Avonex®, Cinnovex®, Betaseron® or Extavia®. Also modifications of interferon e.g. pegylation and the like are within the ambit of the invention.

The inventors have identified several useful markers to determine response to interferon-beta treatment. The inventors were the first to apply the genetic markers in the way according to the invention and in particular in a combination to identify if a MS patient will probably not respond to interferon-beta treatment or show good or superior response to treatment. The method according to the invention gives superior and unexpected good results having implications for diagnostic purposes and for treatment schemes of MS patients. In this way a highly reliable method could be developed to identify the level of response to interferon treatment in a MS patient. In one aspect of the invention a highly predictive method was established for MS patients to be treated with interferon-beta, in particular interferon-beta 1a or 1b. Of particular interest in this context are the drugs marketed as Rebif®, Avonex®, Cinnovex®, Betaseron® and Extavia®.

The invention in one aspect provides for a method of treating a MS patient subgroup with interferon-beta, preferably with interferon-beta 1a or 1b. In this method the MS patient subgroup, i.e. the individual patients, can be selected according to the result of the identified response to interferon-beta treatment. In this manner it is now advantageously possible to safe resources and to refrain from treating patients who were identified as non-responders to interferon-beta treatment. On the other hand it will give physicians and MS patients more security in predicting the success rate of interferon-beta treatment. In particular the selection of MS patients identified as responders or super-responders for treatment with interferon-beta, in particular interferon-beta 1a, especially Rebif®, in view of the cost of treatment has positive implications not only from a treatment perspective but also on an economical dimension. Accordingly, the inventive method of identifying the response to treatment to interferon-beta can be viewed as a means to stratify the treatment of MS patients with interferon-beta, preferably with interferon-beta 1a or 1b, and in particular Rebif®, Avonex®, Cinnovex® Betaseron® and Extavia® as well as comparable interferon-beta products.

“Response” or “responders” to interferon treatment in an individual diagnosed as having MS, suffering from MS or a MS patient in the sense of the present invention is understood to be residual disease activity according to the criteria set out below upon interferon treatment, in particular with interferon-beta 1a or 1b, and in particular Rebif®, Avonex®, Cinnovex® Betaseron® and Extavia®, of a MS patient. The response may be defined and/or measured as increase in time to the progression of the disease as measured by e.g. Expanded Disease Status Scale (EDSS) or with other commonly used techniques or measurements or definitions in the field. In particular it is to be understood as non-progression or non-worsening of MS or a stable clinical profile/activity or as the improvement of MS in e.g. clinical signs or measured with other means as e.g. MRI or CSF (cerebrospinal fluid) analysis. In particular it may be understood as less frequent relapses/attacks/exacerbation or milder relapses/attacks/exacerbation.

The term “non-responders” refers to an individual suffering from MS or a MS patient in the sense of the present invention who in comparison to the average response to interferon treatment exhibit a lower level of response, i.e. exhibit a higher level of residual disease activity according to the criteria set out below upon interferon treatment. It may be understood as no improvement of the disease or a progression or worsening of MS as defined by the analysis of the patient as set out below.

Equally, the term “super-responders” refers to an individual suffering from MS or a MS patient in the sense of the present invention who in comparison to the average response to interferon treatment exhibit a higher level of response, i.e. exhibit a lower level of residual disease activity according to the criteria set out below upon interferon treatment.

The term “residual disease activity” according to the invention is to be understood as indicating a certain level of MS disease activity, e.g. showing clinical symptoms, as defined by any of the measurements or definitions usually applied in the field of MS. One indicator or measurement of “residual disease activity” can be the experience of relapse(s) or disease progression as e.g. measured by Expanded Disease Status Scale (EDSS) or Magnetic Resonance Imaging (MRI). As time frame one example is the assessment during two years of treatment. It is appreciated that other time frames may be defined and used, e.g. one year, three years, or others as usually applied in clinical study protocols and well known to the skilled person. The time frame of reference may be chosen so as to allow for a measurement and appropriate read-out. Equally applicable, other accepted disease status measurements may be applied as e.g. The Cambridge Multiple Sclerosis Basic Score (CAMBS) and others used by the skilled person. There exist various definitions of an MS attack in the field and as understood by the skilled person in the field of MS that may be applied according to the invention. Accordingly, various possibilities exist for the skilled person that can be applied when working the invention. Examples of the assessment or diagnosis of MS are published in Kurzke J. F., Neuroepidemiology, 1991, 10: 1-8 (8); Kurzke J. F., Neurology, 1983, 33: 1444-1452 (7); McDonald W. I et al., Ann. Neurol., 2001, 50: 121-127 (9); Polman C. H. et al., Ann. Neurol. 2005, 58: 840-846 (10).

As used in the specification and the claims, “a” or “an” means one or more unless explicitly stated otherwise.

An “allele” is a particular form of a gene, genetic marker or other genetic locus, that is distinguishable from other forms of the gene, genetic marker or other genetic locus; e.g. without limitation by its particular nucleotide sequence. The term allele also includes for example without limitation one form of a single nucleotide polymorphism (SNP). An individual can be homozygous for a certain allele in diploid cells; i.e. the allele on both paired chromosomes is identical; or heterozygous for said allele; i.e. the alleles on both paired chromosomes are not identical.

A “genetic marker” is an identifiable polymorphic genetic locus. An example without limitation of a genetic marker is a single nucleotide polymorphism (SNP). A “marker” may be a genetic marker or any other marker, e.g. the expression level of a particular gene on nucleotide level as mRNA, useful in the context of the invention to be indicative of a response to interferon treatment.

A “genotype” as used herein refers to the combination of both alleles of a genetic marker, e.g. without limitation of an SNP, on a single genetic locus on paired (homologous) chromosomes in an individual. “Genotype” as used herein also refers to the combination of alleles of more than one genetic loci, e.g. without limitation of SNPs, on a pair or more than one pair of homologous chromosomes in an individual.

“Genotyping” is a process for determining a genotype of an individual.

“Locus” or “genetic locus” refers to a specific location on a chromosome or other genetic material.

“Oligonucleotide” refers to a nucleic acid or a nucleic acid derivative; including without limitation a locked nucleic acid (LNA), peptide nucleic acid (PNA) or bridged nucleic acid (BNA); that is usually between 5 and 100 contiguous bases in length, and most frequently between 5-40, 5-35, 5-30, 5-25, 5-20, 5-15, 5-10, 10-50, 10-40, 10-30, 10-25, 10-20, 15-50, 15-40, 15-30, 15-25, 15-20, 20-50, 20-40, 20-30 or 20-25 contiguous bases in length. The sequence of an oligonucleotide can be designed to specifically hybridize to any of the allelic forms of a genetic marker; such oligonucleotides are referred to as allele-specific probes. If the genetic marker is an SNP, the complementary allele for that SNP can occur at any position within an allele-specific probe. Other oligonucleotides useful in practicing the invention specifically hybridize to a target region adjacent to an SNP with their 3′ terminus located one to less than or equal to about 10 nucleotides from the genetic marker locus, preferably ≦about 5 nucleotides. Such oligonucleotides hybridizing adjacent to an SNP are useful in polymerase-mediated primer extension methods and are referred to herein as “primer-extension oligonucleotides.” In a preferred embodiment, the 3′-terminus of a primer-extension oligonucleotide is a deoxynucleotide complementary to the nucleotide located immediately adjacent an SNP.

“Polymorphism” refers of two or more alternate forms (alleles) in a population of a genetic locus that differ in nucleotide sequence or have variable numbers of repeated nucleotide units. Polymorphisms occur in coding regions (exons), non-coding regions of genes or outside of genes. The different alleles of a polymorphism typically occur in a population at different frequencies, with the allele occurring most frequently in a selected population sometimes referenced as the “major” or “wildtype” allele. Diploid organisms may be homozygous or heterozygous for the different alleles that exist. A biallelic polymorphism has two alleles.

In particular three markers, i.e. IFNAR2-rs4986956, PSMB8-rs2071543 and MX1-rs469304, were identified as useful either alone or in combination in the method according to the invention. IFNAR2-rs4986956 has been identified as predictive as well as its combination with PSMB8-rs2071543 and MX1-rs469304, respectively. In particular with the combination of the markers according to the invention a very positive and superior result in diagnosing the response to interferon, in particular interferon-beta and specially interferon-beta 1a, could be achieved.

The invention in particular provides advantageously for a method that can be used to identify the “super-responders” to interferon treatment in MS patients. “Super-responders” refers to those individuals who can be identified to show improved response to interferon treatment, in particular interferon-beta and especially interferon-beta 1a, in comparison to the MS population who exhibit an average response level upon interferon treatment. The “super-response” is exhibited by reduced residual disease activity according to the criteria as set out and defined herein. The invention thus provides for the identification of the level of response to interferon-beta, i.e. to identify “responders”, “non-responders” and “super-responders”.

The inventors have developed a method wherein it is possible as illustrated by the examples to identify the following:

Applying the marker PSMB8-rs2071543 (GT group) in one aspect of the method according to the invention it was possible to identify:
Frequency: 20% of the population (matches the frequency observed in general caucasian population);
Response rate: 92% of responders;
An additional relative 19.2-40.7% decrease in relapse rate at 2 years could be obtained when compared to the overall population.

Applying the marker IFNAR2-rs4986956 (TT group) in one aspect of the method according to the invention it was possible to identify:
Frequency: 80% of the population (matches the frequency observed in general caucasian population);
Response rate: 67% of responders;
An additional relative 5.6-9.3% decrease in relapse rate at 2 years could be obtained when compared to the overall population.

The marker IFNAR2-rs4986956 exists in three different form in the population TT, CT or CC, the less frequent form is CC. MS patients with the TT form have more chances to have a good response to interferon-beta treatment (67% of this responder group), compared to the general population. Patients with the CT form have reduced chances to have a good response to Rebif treatment (33% of this responder group) but still respond and patients with the CC form do not respond to interferon-beta treatment.

The marker PSMB8-rs2071543 exists in two or three forms in the population GG, CT and TT, the last form is the less frequent (either absent of less than 1.7% of the population). Patients with the GT form have the very high chances to respond well to interferon-beta treatment (93% of responders) while patients with the GG form have a slight decrease chance of good response (50% of responders) when compared to the entire population.

The marker MX1-rs469304 exists as TT, CT or CC. MS patients with the CC form have a high chance to respond well to interferon-beta treatment. Those exhibiting the CT form respond equally well when compared to the entire population.

A, T, C and G represent adenine, thymine, cytosine and guanine, respectively.

In addition, patients carrying both the PSMB8-rs2071543 GT form and the IFNAR2-rs4986956 TT or CT forms have 100% of chances to have a good response to interferon-beta treatment after e.g. two years. Also a combination of the markers MX1-rs469304 and IFNAR2-rs4986956 shows an elevated level of treatment response rate.

The analysis can be performed with state of the art methods of genotyping known by the person skilled in the art, e.g. as illustrated by Wang et al. (6).

There is limited knowledge of the effect of genetic on interferon-beta treatment. Prior publications have shown that patients with the PSMB8-rs2071543 GT form would respond less to interferon treatment without specificity toward a sub-type of interferon. The inventors show for the first time IFNAR2-rs4986956 and its impact on interferon response. In addition, the inventors show that the combination of the PSMB8-rs2071543 GT form with IFNAR2-rs4986956 TT can be used to predict an association with 100% of chance of response to interferon-beta after e.g. two years of treatment. Also a combination of IFNAR2-rs4986956 and MX1-rs469304 advantageously can be used to predict the level of response to treatment with interferon in an individual having MS. Accordingly, this method of identifying the response to treatment can be applied in a method of treating MS patients with an interferon-beta as defined herein in a stratified manner and for treatment of a MS patient subpopulation.

It will be appreciated that these same patients who show response to interferon-beta 1a will have a similar response to interferons in general. The markers according to the invention are most predictive for interferon-beta. In one embodiment the prediction can be made for interferon-beta 1a. In another embodiment the prediction can be made for interferon-beta 1b. In particular predictive are the markers according to the invention for Rebif®, Avonex®, Cinnovex® or Betaseron® and Extavia®.

In one embodiment the method of the invention is indicative of a good response level to interferon treatment wherein in IFNAR2-rs4986956 is T/T, in PSMB8-rs2071543 is G/T and in MX1-rs469304 is C/C.

In another embodiment the invention relates to a kit for detecting a genetic marker that is associated with the level of response to interferon, the kit comprising a probe or a set of oligonucleotides (primers) designed for identifying each of the alleles at each polymorphic site (PS) in the marker. In such a kit the probe or set of primers is specific for IFNAR2-rs4986956, PSMB8-rs2071543 and/or MX1-rs469304.

In another aspect of the invention it is also possible to use a combination of the above mentioned SNPs and/or to measure with standard techniques known by the person skilled in the art the expression level of the genes relating to IFNAR2, PSMB8 and/or MX1. In another alternative embodiment a combination of SNP(s) and the expression level of the genes relating to IFNAR2, PSMB8 and/or MX1 can be applied to identify the response to interferon treatment.

In yet another aspect the invention relates to a method for treating Multiple Sclerosis in an individual in need thereof, the method comprising the steps i. identifying response to interferon-beta treatment according to the method of identifying as pointed out above; ii. treating the individuals identified as non-responders, responders or super-responders with interferon-beta as appropriate according to their genetic status.

One advantage of the method of treatment according to the invention is that the treatment can be selected according to the patients identified genotype and genetic markers. In this manner the treatment can be adapted to each individual patient if considered advantageous.

In one aspect, the invention provides for the use of an interferon-beta for the preparation of a medicament for preventing or treating MS in an individual wherein the individual exhibits one of the genetic markers or a combination thereof or by way of the expression markers on e.g. nucleic acid level as described above. In this aspect the genetic markers are preferably determined by the diagnostic method according to the invention as described above.

In one aspect, the invention provides for an interferon, preferably an interferon-beta, for use to prevent or treat MS in an individual wherein the individual has been diagnosed before treatment to exhibit one of the markers or a combination thereof selected from IFNAR2-rs4986956, PSMB8-rs2071543 and MX1-rs469304, preferably wherein in IFNAR2-rs4986956 thymine/thymine, in PSMB8-rs2071543 guanine/thymine and/or in MX1-rs469304 cytosine/cytosine is present. Preferably, the diagnosis is performed by a method described herein.

In one aspect, the invention provides interferon-beta for use to prevent or treat MS in an individual wherein the individual exhibits one of the markers or a combination thereof as described above.

In the method of identifying, kit or method of treating according to the invention the interferon-beta is preferably interferon-beta 1a or 1b. Particular embodiments of the invention refer to interferon-beta that is Rebif®, Avonex®, Cinnovex® or Betaseron® and Extavia®.

One advantage of the method of treating or the use of interferon-beta according to the invention is that it is now possible to adapt the MS treatment more closely to the need of the individual patient. In particular, the inventors now provide a method of treatment or use of interferon-beta in a manner to stratify MS treatment and target a particular patient population according to their genetic background. Thus, it is advantageously possible by application of the method according to the invention to individuals suffering from MS to target individuals who will respond to interferon-beta treatment. Patients who know that they will respond to a treatment will show a better compliance with therapy, which in turn will positively impact on the therapy. The application of the invention accordingly has positive implications on various levels, e.g. positive treatment results, cost saving aspects and more effective use of resources in the medical sector.

In another aspect it may be possible to adapt the treatment scheme as to the route of application and adaptation of the drug dosage as may be required to stratify the treatment according to the particular patients needs. This may include lowering or increasing the dosage of interferon as appropriate in the particular patient group with regard to their genotype.

In particular useful in the present method of treatment are formulations and dosages as disclosed in WO 95/31213 and WO 2004/096263 being hereby included by reference.

Formulations useful in a method of treating a MS patient according to the invention may be a liquid pharmaceutical formulation comprising interferon-beta and a polyol, a non-reducing sugar or an amino acid as stabilising agent. The stabilising agent may be selected from: mannitol, saccharose and glycine. In a preferred embodiment the stabilising agent is mannitol. The interferon-beta may be from any source wherein a preferred form is recombinant. The interferon-beta may be in a quantity between 0.6 and 1 MIU/ml. The liquid pharmaceutical formulation useful in the present invention may comprise, furthermore, a buffer solution capable of maintaining the pH of the liquid formulation at a value between 3.0 and 4.0. The buffer solution may be an acetate buffer in which the buffer solution preferably has a concentration of 0.01 M. It may contain human albumin. In a preferred embodiment used the pharmaceutical formulation comprises 1 MIU/ml of interferon-beta, 54.6 mg/ml of mannitol, 0.5 mg/ml of albumin in a solution of 0.01 M acetate buffer at pH 3.5. Presentation forms of the liquid pharmaceutical formulation may be hermetically sealed in sterile conditions in a container appropriate for storage prior to use.

Another pharmaceutical formulation used in the present invention may be a stabilized HSA-free liquid pharmaceutical composition comprising an interferon-beta (IFN-beta), wherein said formulation is a solution that comprises a buffer, a surfactant, an isotonicity agent and an anti-oxidant. The IFN-beta is preferably human recombinant IFN-beta. The buffer preferably used is present in an amount sufficient to maintain the pH of said composition within plus or minus 0.5 units of a specified pH, where the specified pH is about 3.0 to about 5.0. In particular useful the pH is 3.5±0.2 or 4.5±0.2. The buffer may be present at a concentration of about 5 mM to 500 mM, preferably at a concentration of about 10 mM. The buffer preferably is acetate buffer, and the isotonicity agent is mannitol. Preferably the isotonicity agent is present at a concentration of about 0.5 mg/ml to about 500 mg/ml, more preferably at a concentration of about 55 mg/ml. The surfactant may be selected from Pluronic® F77, Pluronic F87, Pluronic F88 and Pluronic F68 and preferably the surfactant is present at a concentration of about 0.01 mg/ml to about 10 mg/ml, preferably at a concentration of about 1 mg/ml. The antioxidant may be methionine present at a concentration of about 0.01 to about 5.0 mg/ml, preferably at a concentration of about 0.1 mg/ml. The interferon-beta is present at a concentration of about 10 μg/ml to about 800 μg/m, preferably at a concentration of about 22, 44, 88 or 264 μg/ml. Useful are aqueous solutions. Also the inclusion of a bacteriostatic agent may be considered. Preferably the bacteriostatic agent is benzyl alcohol. Preferably the bacteriostatic agent is present at a concentration of about 0.1% to about 2.0%, preferably at a concentration of about 0.2 or 0.3%. The interferon-beta formulation may be filled in a container hermetically sealed in conditions sterile and appropriate for storage prior to use. The container may be a pre-filled syringe for mono-dose administration or a vial or a cartridge for an auto-injector. Such a formulation may be used as mono-dose or multi-dose administration.

In one aspect the dosage regime may be adapted to the particular outcome of the diagnostic analysis of the markers according to the invention and the method for diagnosing. In particular, before the treatment a diagnostic test according to the invention is performed. More particularly, the genotype of an individual is tested and based upon this result the individual is treated with an interferon as described herein as is advantageous in view of his genotype of one of the markers described in the invention, preferably the treatment scheme is adapted to the genetic status. For example a non-responder may receive a higher dosage, e.g. in the range of 20 to 44 μg/ml, preferably 22 μg/ml, or 44 to 100 μg/ml, preferably 88 μg/ml, depending on the need of the patient. It is also possible to apply a sustained release formulation or slow release formulation of interferon-beta, preferably interferon-beta 1a, in order to adapt the treatment to the identified response to interferon-beta. Such a formulation may be designed as known in the art by the skilled person and will apply release profiles advantageous and required for any particular design.

In the experimentation as performed by the inventors Applied Biosystems TaqMan assays and DNA sequencing technologies for genotyping were used. In general any method on polynucleotide level useful for genotyping may be applied. A statistical analysis was performed in order to compare differences between subgroups of subjects (defined based on clinical measures of response: relapses, EDSS score progression and MRI activity during two years of treatment) of allelic frequencies and genotypic frequencies for each marker independently. Statistical results were classified based on the observed p-value (<0.05) and filtering for minor allelic frequency (<5%) and missing data (>5%). Sub-group comparisons including all defined subgroups were preferentially used to select best markers.

The markers according to the invention were found to be statistically associated and are selected from the group comprising or consisting of PSMB8-rs2071543, MX1-rs469304 and IFNAR2-rs4986956 for interferon response. The preferred interferon for the prediction according to the invention is interferon-beta, preferably interferon-beta 1a, more preferably Rebif® or alternatively Avonex®, or Betaseron® and Extavia®.

When markers were considered independently, all response markers identified in this analysis were risk markers (i.e. no marker was 100% associated with the clinical outcome, except CC genotype for IFNAR2-rs4986956 where all subjects (n=2) were Rebif® low responders), with odd ratios ranging approximately from 3.5 to 22 and with very wide 95% confidence intervals, mainly due to the small sample size. The best response rates observed were approximately 92% of responders for Rebif® (IFNAR2-rs4986956) that represent 23% of the population. The worst response rate observed were 0% of responders to Rebif® for approximately 4% of the PGx population (PSMB8-rs2071543). When markers were combined, the best response rate observed for a subgroup was 100% of responders for the combination of IFNAR2-rs4986956 with PSMB8-rs2071543.

The markers identified associated with interferon response are located in PSMB8, MX1 and IFNAR2 candidate genes. The PSMB8-rs2071543 marker is a change in G to T base at position 188 in mRNA; it induces an amino acid change Lys (K) to Gln (Q) at position 49. PSMB8 (proteasome subunit, beta type, 8 (also known as LMP7—large multifunctional peptidase 7)) is involved in antigen processing and recognition mechanism by lymphocytes. The MX1-rs469304 marker is a synonymous change in T to C base at position 2040 in mRNA; it does not induce an amino acid change. This marker has never been studied before for association with interferon response in multiple sclerosis. The IFNAR2-rs4986956 marker is a change in C to T base at position 351 in mRNA, it induces an amino acid change Ser (S) to Phe (F) at position 8 in amino acid sequence. IFNAR2 is one of the two receptors for interferon alpha and beta.

In the following the present invention shall be illustrated by means of the following examples that are not construed to be viewed as limiting the scope of the invention.

### EXAMPLES

### Targeted Single Nucleotide Polymorphisms Genotyping Using Applied Biosystems TaqMan Assays

The following criteria were used to assess the association between genetic variants and clinical parameters: p-value, odd ratio, relative risk, minor allelic frequency, missing data. Statistical results were classified based on the observed p-value (<0.05) and filtering for minor allelic frequency (<5%) and missing data (>5%). Sub-group comparisons including all defined subgroups were preferentially used to select best markers.

The clinical parameters used were: EDSS, Magnetic Resonance Imaging, Relapse rate, demographic parameter (sex, age, and ethnicity). As statistical methods Chi-square was applied.

### Sample Preprocessing

Blood samples for DNA extraction were collected in EDTA tubes and stored at −70° C. until processing.

DNA was extracted from whole blood using QIAgen kit. After extraction, DNA samples absorbance was measured at wavelengths of 260 nm and 280 nm using a spectrophotometer and gel electrophoresis on agarose gels were performed to estimate the quantity and quality of DNA.
260 nm/280 nm absorbance ratio and DNA concentration calculated from the 280 nm absorbance measure using a spectrophotometer correspond to the first quality control (QC.1).
Electrophoresis on agarose gel corresponds to the second quality control (QC.2).

All DNA samples passed the acceptance criteria defined for QC.1 (absorbance ratio was between 1.7 and 2.0 and DNA concentration was above 50 ng/4).

All DNA samples passed the acceptance criteria defined for QC.2 (for each sample, one band was visible on agarose gel after electrophoresis at a high molecular weight corresponding to non-degraded genomic DNA).

An aliquot from each sample was distributed into 2 micro plates (96 wells), numbered 50-PL1 (containing 60 samples) and 50-PL2 (containing 53 samples). Each micro-plate contained also a negative control and a reference genomic DNA “103”.

The micro-plates were transferred for genotyping (TaqMan SNP genotyping).

### TaqMan SNP Genotyping

TaqMan SNP genotyping was performed to detect the selected markers.

Assays were obtained from Applied Biosystems, by submitting the SNP reference number (i.e. rs number). Upon receipt, the reaction was prepared following the specification of the vendor (Applied Biosystems). Briefly, the reaction mixture was prepared by mixing 2 μL of the purified DNA solution with the reaction mix containing the probe set provided by Applied Biosystems. The reaction was performed by Polymerase Chain Reaction (PCR) in a thermocycler, using the parameters provided by Applied Biosystems. The genomic DNA samples and nineteen no template controls (NTCs) were distributed in two quadrants of one 384-wells plate. 384-well plates, containing the same genomic DNA sample distribution, were prepared in one time for all TaqMan SNP assay.

For each TaqMan SNP assay, the reaction mix containing the PCR master mix and the SNP assay (primers and probes) was added into each reaction well and PCR amplification was run using MJ Research PTC-225 tetrad DNA engine.

After PCR cycles, plate was read using ABI 7900HT instrument then analyzed with the End Point Allelic Discrimination method.

The following quality controls were applied to determine the genotype of the markers:

Distinct NTC cluster with no fluorescent signal (lower left hand corner of the plot) and no more than one NTC with determined call.

Distinct sample clusters corresponding to specific allele types (homozygous allele 1, heterozygous allele 1 & 2, homozygous allele 2).

Less than fifteen percent of samples undetermined.

For the TaqMan SNP assays, all acceptance criteria of defined above were reached.

The principle of the detection assay is described below.

In a TaqMan® SNP Genotyping assay, two locus-specific PCR primers that surround the SNP were used to amplify fragment of approximately 100 bp. Two allele-specific probes were used to detect the two SNP allele bases. At the 5′ end of one probe was a fluorescent reporter dye, FAM, and a second probe has the VIC reporter dye. Each probe also has a non-fluorescent quencher dye, MGB, at the 3′ end. As DNA was amplified in each PCR cycle, if the target sequence of the allele-specific probe was amplified, the probe did hybridize to the DNA during the annealing step and extended. When the DNA polymerase molecules came into contact with this hybridized probe, the reporter dye of the probe was cleaved from the probe leaving the quencher dye behind. In each cycle of the PCR, cleavage of the reporter dyes from one or both of the allele-specific probes caused an exponential increase in the fluorescent intensity. After PCR, the total fluorescence of each sample was read on the ABI 9700 (384-well format). If fluorescence is observed from only one probe, the sample was homozygous for that allele the probe was specific for. If fluorescence was observed for both allele-specific probes, the sample is heterozygous for both alleles. If the probe did not hybridize, the fluorescence of the dye was “quenched”, or reduced, by the quencher dye, and thus minimal fluorescence was observed, indicating a failed genotype.

### Statistics

Association of SNPs with the phenotypic traits considered was assessed using a p-value calculated with an exact Fisher tests (testing the null hypothesis that differences in allele and genotype distribution between the two phenotypic groups are due only to sampling fluctuation, the two groups being drawn from the same population).

SNPs that did not meet criteria of minimal call rate (missing data proportion >5%) and minimal frequency of polymorphism (minor allele frequency <5%) were filtered out prior to this analysis.

Input data set for a given pair of phenotypic groups was provided as genotypic contingency tables. For each marker, p values and odd ratios with their confidence intervals at 95% were estimated using the FREQ procedure from the SAS software (release 9.1.3, service pack 4). No correction for multiple testing was performed.

### Clinical Variables Used for the Analysis of Interferon Response

55 individuals were treated with Rebif®. Response to Rebif® was defined using the following categories:

### Group 1:

No relapses and no EDSS progression during 96 weeks of study treatment and no MRI activity

### Group 2:

No relapses and no EDSS progression during 96 weeks of study treatment, but positive evidence of MRI activity evidenced by at least two of the following MRI parameters:

New T1 Gd-enhancing lesions
New T2 lesions
Enlarging T2 (burden of disease)
New T1 hypointense lesions
Enlarging T1 hypointense lesions
Brain volume changes

### Group 3:

One relapse and no EDSS progression during 96 weeks of study treatment
OR
   No relapse and 0<EDSS progression <1

### Group 4:

>1 (2 or more) protocol-defined (qualifying) relapses during 96 weeks of study treatment
OR
   >1 EDSS point progression, sustained over at least 3 months during 96 weeks of study treatment

### Group 5:

>1 (2 or more) protocol-defined (qualifying) relapses during 96 weeks of study treatment
AND
   >1 EDSS point progression, sustained over at least 3 months during 96 weeks of study treatment

Other examples selected from a number of possibilities to defining the residual disease activity and defining the MS status are:

“All the following criteria (a, b, c) are to be met for establishing an MS clinical attack:
a) Neurological abnormality, either newly appearing or re-appearing, with abnormality specified by both
i) Neurological abnormality separated by at least 30 days from onset of a preceding clinical event,
and
   ii) Neurological abnormality lasting for at least 24 hours
   b) Absence of fever or known infection (fever with temperature (axillary, orally or intrauriculary)>37.5° C./99.5° F.)
   c) Objective neurological impairment, correlating with the subject's reported symptoms, defined as either
   i) Increase in at least one of the functional systems of the EDSS
or
   ii) Increase of the total EDSS score
   The occurrence of paresthesia, fatigue, mental symptoms, and/or vegetative symptoms without any additional symptom will not be classified as an MS clinical attack.”
   or

“A qualifying relapse (or attack) is defined as a new or worsening neurological symptom, in the absence of fever, lasting for 24 hours, accompanied by an objective change in the relevant (i.e. symptomatic) functional system, and preceded by at least 30 days of clinical stability or improvement. It is possible, due to resolution of impairment related to a prior relapse or intra/inter-observer variability on EDSS assessment, that the EDSS may not change or may even improve despite worsening in the relevant functional system.

A non-qualifying relapse would be any relapse that did not have the requisite neurological changes as above. This could include, for example, an amnestic relapse that was not assessed while ongoing or an ongoing relapse but without a change in EDSS/functional system but, for either scenario, with convincing historical features of focal neurological dysfunction consistent with a relapse in the opinion of the assessor.”

or
according to J. F. Kurtzke (7).

### IFNAR2 Gene

One SNP (IFNAR2-rs4986956) shows significant association with Rebif® response based on our selection criteria.

The tables and Figures provide the distribution of genotypes between good responders (groups 1 and 2) and poor responders (groups 3, 4 and 5).

The SNP has the following characteristics:

### Presence of T:

sensitivity marker good responders=100%
specificity marker good responders=9%
sensitivity marker poor responders=91%
specificity marker poor responders=0%

### Presence of C:

sensitivity marker good responders=8%
specificity marker good responders=65%
sensitivity marker poor responders=35%
specificity marker poor responders=92%
   The response rate for each genotype category is as follow (compared to 60% in the total PGx population):

| TT | CT | CC |
|---|---|---|
| 67% | 33% | 0% |

The response rate for the presence or absence of a given allele is:
For presence of T allele: 62%
For the absence of T allele: 0%
For the presence of C allele: 27%
For the absence of C allele: 67%
The frequencies of these genotypes in the total the PGx sample population is:

| TT | CT | CC |
|---|---|---|
| 80% | 16% | 4% |

The frequency of the C allele in the total PGx sample population is: 89%
The frequency of the presence of the C allele in the PGx sample population is: 19%
The frequency of the absence of the C allele in the PGx sample population is: 81%
The frequency of T allele in the PGx sample population is: 11%
The frequency of the presence of the T allele in the PGx sample population is: 96%
The frequency of the absence of the T allele in the PGx sample population is: 4%

### MX1 Gene

There is a significant SNP (MX1_rs469304), response based on our selection criteria.
The SNP has the following characteristics:

### Presence of T:

sensitivity marker good responders=69%
specificity marker good responders=17%
sensitivity marker poor responders=83%
specificity marker poor responders=35%

### Presence of C:

sensitivity marker good responders=80%
specificity marker good responders=43%
sensitivity marker poor responders=57%
specificity marker poor responders=20%
   The response rate for each genotype, category is as follow (compared to 60% in the total PGx population):

| TT | CT | CC |
|---|---|---|
| 41% | 62% | 75% |

The response rate for the presence or absence of a given allele is:
For presence of T allele: 54%
For the absence of T allele: 75%
For the presence of C allele: 67%
For the absence of C allele: 41%
The frequencies of these genotypes in the total the PGx sample population is:

| TT | CT | CC |
|---|---|---|
| 30% | 42% | 28% |

The frequency of the C allele in the total PGx sample population is: 49%
The frequency of the presence of the C allele in the PGx sample population is: 70%
The frequency of the absence of the C allele in the PGx sample population is: 30%
The frequency of T allele in the PGx sample population is: 51%
The frequency of the presence of the T allele in the PGx sample population is: 72%
The frequency of the absence of the T allele in the PGx sample population is: 28%

### PSMB8 Gene

PSMB8 candidate gene results association with response to treatment with Rebif®, for the various groups response comparisons.

One SNP was significantly associated with Rebif® response (PSMB8 rs2071543) based on our selection criteria.

The SNP has the following characteristics:

### Presence of T:

sensitivity marker good responders=100%
specificity marker good responders=0%
sensitivity marker poor responders=100%
specificity marker poor responders=100%

### Presence of G:

sensitivity marker good responders=35%
specificity marker good responders=96%
sensitivity marker poor responders=4%
specificity marker poor responders=65%
   The response rate for each genotype category is as follow (compared to 60% in the total PGx population):

| GG | GT | TT |
|---|---|---|
| 50% | 92% | 0% |

The response rate for the presence or absence of a given allele is:
For presence of G allele: 60%:
For the absence of G allele: 0%
For the presence of T allele: 92%
For the absence of T allele: 50%
The frequencies of these genotypes in the total the PGx sample population is:

| GG | GT | TT |
|---|---|---|
| 77% | 23% | 0% |

The frequency of the G allele in the total PGx sample population is: 89%
The frequency of the presence of the G allele in the PGx sample population is: 100%
The frequency of the absence of the G allele in the PGx sample population is: 0%
The frequency of T allele in the PGx sample population is: 11%
The frequency of the presence of the T allele in the PGx sample population is: 23%
The frequency of the absence of the T allele in the PGx sample population is: 77%

TABLE 1
distribution of response groups

| | #patients treated |
|---|---|
| Response group | with Rebif |
| 1 | 23 |
| 2 | 10 |
| 3 | 12 |
| 4 (+5) | 11 |
| total | 56 |

TABLE 2
IFNAR2 candidate gene results association with response
to treatment with Rebif ® (Groups 2 vs. 3)

| Marker | N1 | N2 | allOR | LowerCL | UpperCL | ChiSqGen | ChiSqAll | pgeno | pall |
|---|---|---|---|---|---|---|---|---|---|
| IFNAR2_rs4986956 | 10 | 12 | 0.00 | . | . | 4.07 | 4.70 | 0.13 | 0.05 |

N1: number of patients bellowing to clinical definition 1 (here clinical definition group 2)
N2: number of patients bellowing to clinical definition 2 (here clinical definition group 3)
allOR: allelic Odd Ratio
LowerCL: Lower limit of the 95% confidence interval of the allelic odd ratio
UpperCL: Upper limit of the 95% confidence interval of the allelic odd ration
ChiSqGen: Chi Square Genotypic
ChiSqAll: Chi Square Allelic
pgeno: p-value genotypic

Pall: p-value allelic
(The definitions above are applicable for all the tables below)

TABLE 3
IFNAR2 candidate gene results association with response to treatment
with Rebif ® (Groups 1 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| IFNAR2_rs4986956 | 24 | 23 | 0.24 | 0.06 | 0.94 | 3.98 | 4.73 | 0.12 | 0.04 |

TABLE 4
IFNAR2 candidate gene results association with response to treatment with
Rebif ® (1 + 2 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| IFNAR2_rs4986956 | 34 | 23 | 0.17 | 0.04 | 0.64 | 6.69 | 8.15 | 0.023 | 0.003 |

TABLE 5
IFNAR2 candidate gene results association with response to treatment
with Rebif ® (1 + 2 vs. 4 + 5)

| Marker | N1 | N2 | allOR | LowerCL | UpperCL | ChiSqGen | ChiSqAll | pgeno | pall |
|---|---|---|---|---|---|---|---|---|---|
| IFNAR2_rs4986956 | 34 | 11 | 0.16 | 0.03 | 0.72 | 5.96 | 6.89 | 0.042 | 0.019 |

TABLE 6
IFNAR2 candidate gene results association with response to treatment
with Rebif ® (2 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| IFNAR2_rs4986956 | 10 | 23 | 0.00 | . | . | 4.59 | 5.12 | 0.16 | 0.02 |

TABLE 7
MX1 candidate gene results association with response to treatment with Rebif ® (2 vs. 3)

| Marker | N1 | N2 | allOR | LowerCL | UpperCL | ChiSqGen | ChiSqAll | pgeno | pall |
|---|---|---|---|---|---|---|---|---|---|
| MX1_rs469304 | 10 | 12 | 3.10 | 0.90 | 10.65 | 5.40 | 3.30 | 0.07 | 0.13 |

TABLE 8
MX1 candidate gene results association with response to treatment with
Rebif ® (1 + 2 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| MX1_rs469304 | 34 | 23 | 2.29 | 1.06 | 4.94 | 4.06 | 4.57 | 0.15 | 0.03 |

TABLE 9
MX1 candidate gene results association with response to treatment
with Rebif ® (2 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| MX1_rs469304 | 10 | 23 | 3.17 | 1.06 | 9.49 | 6.24 | 4.42 | 0.05 | 0.05 |

TABLE 10
PSMB8 candidate gene results association with response to treatment with Rebif ® (2 vs. 3)

| Marker | N1 | N2 | allOR | LowerCL | UpperCL | ChiSqGen | ChiSqAll | pgeno | pall |
|---|---|---|---|---|---|---|---|---|---|
| PSMB8_rs2071543 | 10 | 12 | 0.00 | . | . | 7.76 | 6.77 | 0.01 | 0.01 |

TABLE 11
PSMB8 candidate gene results association with response to treatment
with Rebif ® (1 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| PSMB8_rs2071543 | 24 | 23 | 0.13 | 0.02 | 1.10 | 5.12 | 4.65 | 0.04 | 0.07 |

TABLE 12
PSMB8 candidate gene results association with response to treatment with
Rebif ® (1 + 2 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| PSMB8_rs2071543 | 34 | 23 | 0.10 | 0.01 | 0.83 | 7.46 | 6.50 | 0.01 | 0.01 |

TABLE 13
PSMB8 candidate gene results association with response to treatment
with Rebif ® (2 vs. 3 + 4 + 5)

| Marker | N1 | N2 | allOR | LowerCL | UpperCL | ChiSqGen | ChiSqAll | pgeno | pall |
|---|---|---|---|---|---|---|---|---|---|
| PSMB8_rs2071543 | 10 | 23 | 0.07 | 0.01 | 0.62 | 9.76 | 8.79 | 0.01 | 0.01 |

TABLE 14
Genetic markers

| | | | Genomic | |
|---|---|---|---|---|
| Marker | Allele 1 | Allele 2 | locus | Flanking sequences |
| rs2229207 | C (forward) | T (forward) | IFNAR2 | CCCTAACTGA TATGTCCTTT GTCTAGCAAT TCTACTTCTG GAAATTTTCC |
| (formerly | G (reverse) | A (reverse) | | CACACTTAAA CTGGCACATG TGCAAAATGA AAGAGATTTC CTTGCAGAAG |
| called | | | | AGACTGGAAA TAATTCAAAT GTCTATAGTA GGGGTACTAA TTATATAAAT |
| rs4986956) | | | | AATGGTGTGT CTATAGAGTA GAACTGTATC CAGCTATTTA AAAAACAAGG |
| | | | | ATAGAAAGAT CTTCCAAACA TATTTTTAAG TGGAAAAAGC AGGGTGGAAC |
| | | | | AGGGACTATA CATGCTATCT CTTGTCTAAA AGATGGGGAA ATTAGGAATA |
| | | | | TGTATTTGTT TTTGCTTATG CATGAAGATA CTCTGAAGGA TTCACAAAAG |
| | | | | AAAAAAAAGT TTTCTAAAGG GAGCAAGACT TTTCACAATT TTTTTTTNGA |
| | | | | ATGCTTAAAA TTGTTGAACC CAACGACTGT TGCCTGTTCA AAAGTTAAAT |
| | | | | TTAAAATTCC TCCCAGACTA GGTAGGGGAT TGGGCCCAGA AGCTGAGACC |
| | | | | AGGCTCACTT GAATAAATGG GGTAAATGAC TAGGAATTTT ACTATTCCTT |
| | | | | ACAGGTCTCT CATTATCTTG TCTTTGCTCC CATTTTTATA TTTGCAGTTT |
| | | | | AATTAGACAC TTCAGAATTT TGATCACCTA ATGTTGATTT CAGATGTAAA |
| | | | | AGTCAAGAGA AGACTCTAAA AATAGCAAAG ATGCTTTTGA GCCAGAATGC |
| | | | | CT |
| | | | | Y |
| | | | | CATCKTCAGA TCACTTAATT TGGTTCTCAT GGGTAAGTGC TGCTTTTTAT |
| | | | | CTTAGCTCTT |
| | | | | ATAGAAGCAA GCTGTGATGC CATCCTCA |
| rs469304 | A (forward) | G (forward) | MX1 | TATTTTTTAC TGTAGTCCAA AATTGAAGAC ATTAGAGCAG AACAAGAGAG |
| | T (reverse) | C (reverse) | | AGAAGGTGAG AAGCTGATCC GCCTCCACTT CCAGATGGAA CAGATTGTCT |
| | | | | ACTGCCAGGA CCAGGTATAC AGGGGTGCAT TGCAGAAGGT CAGAGAGAAG |
| | | | | GAGCTGGAAG AAGAAAAGAA GAAGAAATCC TGGGATTTTG GGGCTTTCCA |
| | | | | R |
| | | | | TCCAGCTCGG CAACAGACTC TTCCATGGAG GAGATCTTTC AGCACCTGAT |
| | | | | GGCCTATCAC CAGGTACGTC TTCGCGTGGT TCAGGATGCC AGCTTCCATT |
| | | | | CTTTCCTTTT CTTCTGAACG CCTCTCTCTT TAGTCTTGCT CTCTCTGTAG |
| | | | | GTGACGTTGG TCAGCTCTGT CGTTTACCTC CTTGTTAGCC TCCTGTATTA |
| | | | | GTCCATTTTC ATGCTGCTGA TAAAGACATA CCTGAGACTG GGCAATTTAC |
| | | | | AAAAGAAAGA GGTTTAACGG ACTTACAGTT CCACATGGCT GGGGAGGCCT |
| | | | | TCTACCATCA CGGCAGAAGG CAATGGGCAC TTCTTACCTG GCGGCGGTGG |
| | | | | CAAGAGAGAG AATGAGAGCC AAGTGAAAGG GGTTTCCCCT TATCAAACCA |
| | | | | TCAGATCTCA TGAGAGTTAC TCACTACCAT GAGAACAGTA TTGGAGAAAC |
| | | | | TGCCCCCATG ATTCAGTTAT CTCCCCCTGA GTCCCTCCCA CAACAGGTGG |
| rs2071543 | T (forward) | G (forward) | PSMB8 | AGTGGGCTTT TGAAGGCGCC TGTTCCTTTC TCGCTTTCTT CGGAAGACCC |
| | A (reverse) | C (reverse) | (or | TTGACCCATC ATTCCCCCGA CCCCCATAAC GGGAGAGCAG AGAAGCCGGT |
| | | | LMP7) | CCCCAGTGTG ATGGTCCTGG TCCAGGCACT AACTGTCCTT TTCTCGGAAA |
| | | | | AGGCAGGGGG ATTAGGAATA GAGTCTTGTT CCCTCCCCTT CGATCTGTGG |
| | | | | CTTTCGCTTT CACTTCCTCC TCCGAGAGCG GACAGATCTC TGGGTGCTGG |
| | | | | GCGGTCATGG CGCTACTAGA TGTATGCGGA GCCCCCCGAG GGCAGCGGCC |
| | | | | GGAATCGGCT CTCCCGGTTG CGGGAAGCGG GCGTCGCTCG GACCCAGGAC |
| | | | | ACTACAGTTT CTCTATGCGA TCTCCAGAGC TCGCTTTACC CCGGGGAATG |
| | | | | M |
| | | | | AGGTCGGGGC AGTAGGGAAG CCCCTAGGGA TGCAGGGAGG CGGGCGCTGA |
| | | | | GGAGTGGAGG GTCGCCTGAG AGGAGGAGGC GAGAGCGGGA GCGCGGGGTA |
| | | | | CAGGGTCGGG GGTAGCCTTC AGTCCCGGAG AGCGCCAGAC CCAAAGAAGA |
| | | | | GGCCACATGG GGATGGGGCC TGAGAGGAGG AAGTGCAAGT TAGGACAAAG |
| | | | | AGTTACAGGT GAGGTGGGGG CTCCCAAAGG AAGACAAGAG AACTTTCCTG |
| | | | | CCCTTGTCCA CACACAAATG GTGGAGCCTT TTTCATGGGG TTATCACATG |
| | | | | ATATAGGAGG TGTGTGGTGT CTTGGGAAAC CTATGAAATT TGCCTGCTGG |
| | | | | CCTCCTCTCA |
| | | | | GCAACTCACT GTTGCGCGAC TTAGAACAAG TCACTTAGTC |

### LIST OF ABBREVIATIONS

DNA DeoxyriboNucleic Acid
EDSS Expanded Disability Status Scale
Gd Godalinium
HLA Human Leukocite Antigen
HW Hardy-Weinberg
IFNAR2 Type 1 Interferon receptor chain 2
LMP7 Large Multifunctional Proteasome 7
MHC Major Histocompatibility Complex
MRI Magnetic Resonance Imaging
MX1 Myxovirus resistance 1
PGx Pharmacogenetics
RRMS Relapsing Remitting Multiple Sclerosis
SAP Statistical Analysis Plan
SNP Single Nucleotide Polymorphism
N1 number of patients bellowing to clinical definition 1 (here clinical definition group 2)
N2 number of patients bellowing to clinical definition 2 (here clinical definition group 3)
allOR allelic Odd Ratio
LowerCL Lower limit of the 95% confidence interval of the allelic odd ratio
UpperCL Upper limit of the 95% confidence interval of the allelic odd ration
ChiSqGen Chi Square Genotypic
ChiSqAll Chi Square Allelic
pgeno p-value genotypic
Pall p-value allelic

### REFERENCE LIST

(1) Cunningham S, Graham C, Hutchinson M, Droogan A, O'Rourke K, Patterson C, et al. Pharmacogenomics of responsiveness to interferon IFN-beta treatment in multiple sclerosis: a genetic screen of 100 type I interferon-inducible genes. Clin Pharmacol Ther 2005 December; 78(6): 635-46.
(2) Sugimoto Y, Kuzushita N, Takehara T, Kanto T, Tatsumi T, Miyagi T, et al. A single nucleotide polymorphism of the low molecular mass polypeptide 7 gene influences the interferon response in patients with chronic hepatitis C. J Viral Hepat 2002 September; 9(5): 377-84.
(3) Leyva L, Fernandez O, Fedetz M, Blanco E, Fernandez V E, Oliver B, et al. IFNAR1 and IFNAR2 polymorphisms confer susceptibility to multiple sclerosis but not to interferon-beta treatment response. J Neuroimmunol 2005 June; 163(1-2):165-71.
(4) WO 95/31213
(5) WO 2004/096263
(6) Wang, L., Luhm, R., and Lei, M. (2007). SNP and mutation analysis. Adv Exp Med Biol 593:105-116.
(7) J. F. Kurtzke, Neurology 1983: 33, 1444-52.
(8) Kurzke J. F., Neuroepidemiology, 1991, 10: 1-8.
(9) McDonald W. I et al., Ann. Neurol., 2001, 50: 121-127.
(10) Polman C. H. et al., Ann Neurol 2005, 58: 840-846.

1-12. (canceled)

13. A method of identifying the response to treatment with interferon-beta in an individual having Multiple Sclerosis (MS), the method comprising the steps:
a) using a DNA sample of said individual;
b) determining whether in IFNAR2-rs4986956 a thymine or a cytosine is present; or in IFNAR2-rs4986956 a thymine or a cytosine is present and in PSMB8-rs2071543 a guanine or a thymine is present; or in IFNAR2-rs4986956 a thymine or a cytosine and in MX1-rs469304 a thymine or a cytosine is present in the biallelic markers; and
c) correlating the result of step b) with the level of response to the treatment with interferon-beta.

14. The method of claim 13, wherein in IFNAR2-rs4986956 thymine/thymine, in PSMB8-rs2071543 guanine/thymine and in MX1-rs469304 cytosine/cytosine is indicative of a good response level to interferon-beta treatment.

15. A kit for detecting a genetic marker that is associated with the level of response to interferon, the kit comprising a probe or a set of oligonucleotide primers designed for identifying each of the alleles at each polymorphic site (PS) in the marker.

16. The kit of claim 15, wherein the probe or set of oligonucleotide primers is specific for IFNAR2-rs4986956, PSMB8-rs2071543 and/or MX1-rs469304.

17. A method for treating multiple sclerosis in an individual in need thereof, the method comprising the steps:
a) identifying the response to interferon treatment according to claim 13; and
b) treating the individuals identified as responders with an interferon-beta.

18. The method of claim 17, wherein in IFNAR2-rs4986956 thymine/thymine, in PSMB8-rs2071543 guanine/thymine and in MX1-rs469304 cytosine/cytosine is indicative of a good response level to interferon-beta treatment.

19. The method of claim 17, wherein said interferon-beta is interferon-beta 1a or interferon-beta 1b.


SEQUENCE LISTING (3 sequences)

SEQ ID NO: 1 - DNA, 791 bp, Homo sapiens
  misc_feature (398)..(398): n is a, c, g, or t
ccctaactgatatgtcctttgtctagcaattctacttctggaaattttcccacacttaaactggcacatgtgcaaaatgaaagagatttccttgcagaagagactggaaataattcaaatgtctatagtaggggtactaattatataaataatggtgtgtctatagagtagaactgtatccagctatttaaaaaacaaggatagaaagatcttccaaacatatttttaagtggaaaaagcagggtggaacagggactatacatgctatctcttgtctaaaagatggggaaattaggaatatgtatttgtttttgcttatgcatgaagatactctgaaggattcacaaaagaaaaaaaagttttctaaagggagcaagacttttcacaatttttttttngaatgcttaaaattgttgaacccaacgactgttgcctgttcaaaagttaaatttaaaattcctcccagactaggtaggggattgggcccagaagctgagaccaggctcacttgaataaatggggtaaatgactaggaattttactattccttacaggtctctcattatcttgtctttgctcccatttttatatttgcagtttaattagacacttcagaattttgatcacctaatgttgatttcagatgtaaaagtcaagagaagactctaaaaatagcaaagatgcttttgagccagaatgcctycatcktcagatcacttaatttggttctcatgggtaagtgctgctttttatcttagctcttatagaagcaagctgtgatgccatcctca

SEQ ID NO: 2 - DNA, 701 bp, Homo sapiens
tattttttactgtagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccartccagctcggcaacagactcttccatggaggagatctttcagcacctgatggcctatcaccaggtacgtcttcgcgtggttcaggatgccagcttccattctttccttttcttctgaacgcctctctctttagtcttgctctctctgtaggtgacgttggtcagctctgtcgtttacctccttgttagcctcctgtattagtccattttcatgctgctgataaagacatacctgagactgggcaatttacaaaagaaagaggtttaacggacttacagttccacatggctggggaggccttctaccatcacggcagaaggcaatgggcacttcttacctggcggcggtggcaagagagagaatgagagccaagtgaaaggggtttccccttatcaaaccatcagatctcatgagagttactcactaccatgagaacagtattggagaaactgcccccatgattcagttatctccccctgagtccctcccacaacaggtgg

SEQ ID NO: 3 - DNA, 801 bp, Homo sapiens
agtgggcttttgaaggcgcctgttcctttctcgctttcttcggaagacccttgacccatcattcccccgacccccataacgggagagcagagaagccggtccccagtgtgatggtcctggtccaggcactaactgtccttttctcggaaaaggcagggggatgtggaaaagagtcttgttccctccccttcgatctgtggctttcgctttcacttcctcctccgagagcggacagatctctgggtgctgggcggtcatggcgctactagatgtatgcggagccccccgagggcagcggccggaatcggctctcccggttgcgggaagcgggcgtcgctcggacccaggacactacagtttctctatgcgatctccagagctcgctttaccccggggaatgmaggtcggggcagtagggaagcccctagggatgcagggaggcgggcgctgaggagtggagggtcgcctgagaggaggaggcgagagcgggagcgcggggtacagggtcgggggtagccttcagtcccggagagcgccagacccaaagaagaggccacatggggatggggcctgagaggaggaagtgcaagttaggacaaagagttacaggtgaggtgggggctcccaaaggaagacaagagaactttcctgcccttgtccacacacaaatggtggagcctttttcatggggttatcacatgatataggaggtgtgtggtgtcttgggaaacctatgaaatttgcctgctggcctcctctcagcaactcactgttgcgcgacttagaacaagtcacttagtc